(19) United States
(12) Patent Application Publication
(10) Pub. No.: US 2003/0181896 A1
(43) Pub. Date: Sep. 25, 2003

(54) Apparatus and method for cryosurgical treatment of tumors of the breast

(76) Inventors: Roni Zvuloni, Haifa (IL); Uri Amir, Or Yehuda (IL); Doris Schechter, Zikhron (IL); Ravit Barkama, Raanana (IL)

Correspondence Address: Antonhy Castorina, G E Ehrlich, Suite 207, 2001 Jefferson Davis Highway, Arlington, VA, 22202, US

(21) Appl. No.: 10/312,620

(22) PCT Filed:
(86) PCT No.: PCT/IL02/00792

(51) Int. Cl. A61B 18/18

(52) U.S. Cl. 606/20

(57) ABSTRACT

The present invention relates to system, device, and method utilizing cryosurgery to treat a tumor of the breast. More particularly, the present invention relates to treating a breast tumor by inserting into a breast, at a selected site known to be a locus of a tumor, an introducer having at least one access port, operating a biopsy needle through an access port to perform a biopsy of tissues at the selected site, and operating a cryoprobe through an access port to cool body tissues to cryoablation temperatures, thereby ablating or downsizing the tumor. The present invention further relates to use of cryoablation to downsize a large malignant tumor as pre-operative preparation for conventional excision surgery.

### FIELD AND BACKGROUND OF THE INVENTION

The present invention relates to system, device, and method utilizing cryosurgery to treat a tumor of the breast. More particularly, the present invention relates to treating a breast tumor by inserting into a breast, at a selected site known to be a locus of a tumor, an introducer having at least one access port, operating a biopsy needle through an access port to perform a biopsy of tissues at the selected site, and operating a cryoprobe through an access port to cool body tissues to cryoablation temperatures, thereby ablating or downsizing the tumor. The present invention further relates to use of cryoablation to downsize a large malignant tumor as pre-operative preparation for conventional excision surgery.

Breast cancer is the most common type of malignancy occurring in women worldwide.

Despite medical advances of recent years, standard therapeutic responses to breast cancer leave much to be desired. Physically, standard therapies are typically painful, debilitating, and often mutilating as well. Psychologically, fear of disfigurement and loss of femininity resulting from treatment add to the psychological burden associated with cancer diagnoses and cancer treatments of any sort.

Thus, there is a widely felt need for, and it would be highly advantageous to have, a therapeutic approach to benign and malignant breast tumors which speeds, shortens and simplifies clinical treatment of breast tumors tentatively diagnosed as benign, and which speeds, shortens, and simplifies pre-operative treatment of tumors thought to be malignant.

In the case of benign tumors of the breast (e.g., fibroadenomas) and of certain small malignant tumors, ablation of the pathological tissue material IS the therapy. In the case of large malignant tumors, downsizing of the tumor may be an important preliminary step in a multi-step therapeutic process.

With respect to benign tumors, classical excision therapy, removal of a tumor by simply cutting out the offending material, is often not an optimal form of treatment. The preparation, process, and aftermath of classical excision surgery lead to great anxiety and psychological stress in many women. The therapy is frightening, and the recovery is painful. Moreover, surgical excision is likely to cause scarring or other minor or major disfigurement. Breast deformation may result. Further, seen from a generalized social point of view, classical excision surgery as treatment for benign breast tumors is a relatively costly process, generally requiring hospitalization.

Thus there is a widely felt need for, and it would be highly advantageous to have, an apparatus and method for therapeutic treatment of benign breast tumors and of small malignant tumors, which apparatus and method are minimally invasive and are less traumatic than surgical excision, which yield superior cosmetic results when compared to classical excision therapy, and which can be performed as an outpatient procedure.

Not all breast tumors, of course, are benign. Many breast tumors are malignant, and despite efforts at early detection, many malignant tumors are diagnosed when they are in an advanced stage of development.

Early stage tumors have a better prognosis than advanced stage tumors, and are simpler to treat. Advanced stage tumors, typically of larger size and often having lymph node involvement and/or metastases, are considerably harder to treat successfully. Advanced stage tumors require more extensive and complex therapies, and typically do require classical excision surgery.

Even in the case of advanced stage tumors requiring excision, however, pre-operative downsizing of tumors may be used to advantage, reducing tumor volume prior to an excision operation.

There are many advantages to pre-operative downsizing of tumors. Pre-operative downsizing generally facilitates subsequent surgery by rendering the downsized tumor more easily operable. Whereas many advanced-stage tumors, absent pre-operative downsizing, require mastectomy to ensure safe and successful removal, such tumors, after having undergone pre-operative downsizing, may in many cases be safely and successfully removed, including complete tumor excision with a disease-free surgical margin, using breast-conserving techniques such as lumpectomy and quadrantectomy.

Currently accepted expert opinion, based on results of numerous large trials, holds that mastectomy on the one hand, and lumpectomy followed by radiotherapy on the other hand, present equal chances of successful outcome in many cases. Pre-operative downsizing of tumors may serve to increase the practicality of lumpectomy. In general, pre-operative downsizing of a breast tumor makes it more likely that breast-conservation surgery can be undertaken with success, thus improving cosmetic results and preserving a sense of body integrity in the patient.

Pre-operative therapy, moreover, may make surgery a viable option for women whose tumors are considered inoperable due to their seriously advanced stage.

Currently, the available and most commonly practiced methods for pre-operative tumor downsizing are chemotherapy and radiotherapy.

Pre-operative chemotherapy (neoadjuvant chemotherapy) presents a serious medical disadvantage, in that it may prevent accurate subsequent evaluations of tumor pathology. Biopsy samples taken from a patient having undergone pre-operative tumor downsizing by means of pre-operative chemotherapy cannot be relied on to present a reliable clinical picture of tumor status, because pre-operative chemotherapy may change the apparent clinical status of the lymph nodes. Evaluation of lymph nodes as disease-free may lead to an under-estimation of the state of the operated tumor, if pre-operative chemotherapy caused or contributed to that disease-free evaluation. A misleading clinical picture thus produced may result in prescription of inappropriate and inadequate post-operative treatment of the patient, such as prescription for fewer cycles of chemotherapy than are actually needed, or inappropriate choice of drugs.

Of course, a well-informed physician aware that his patent has undergone pre-operative chemotherapy will take into account the above-mentioned ambiguities inherent in pathology reports relating to tissue biopsies performed subsequent to pre-operative chemotherapy. Such a physician will refrain from relying excessively on an apparently “clean” pathology report of his patient's lymph nodes. Thus, dangerously under-proscribed post-operative treatment can be avoided. However, such under-proscription can be avoided only by conservatively over-proscribing of dangerous and debilitating treatments, since in many cases the clean bill of health recited by a pathology report may have in fact reflected a truly non-pathological state in the lymph nodes. In other words, use of pre-operative chemotherapy to down-size breast tumors prior to operations creates, at least, a dangerous ambiguity in subsequent pathology reports, and at worst, a possibility of serious and life-threatening misunderstanding of a patient's clinical state.

Thus there is a widely recognized need for, and it would be highly desirable to have, apparatus and method for pre-operative treatment of malignant tumors of the breast, which treatment results in pre-operative downsizing of tumors yet which does not affect lymph node status, and therefore does not present a danger of masking symptoms and thereby causing a risk of misdiagnosis and inadequate treatment.

An additional problem associated with pre-operative chemotherapy and other classical pre-operative downsizing therapies is that they are generally toxic, and that their toxicity is systemic rather than local. Systemic toxicity of classical pre-operative treatments is extremely unpleasant and debilitating, and causes great suffering among patients.

Thus, there is a widely recognized need for, and it would be highly desirable to have, apparatus and method for pre-operative treatment of malign tumors of the breast, which treatment results in pre-operative downsizing of tumors, yet which does not present systemic toxicity.

Other classical methods of tumor downsizing, such as radiotherapy and biological treatments of various sorts, such as hormonal therapy, also present serious disadvantages. In particular, such treatments typically require multiple visits by a patient to a hospital or clinic, which visits are not only an expensive, tiring, and time-consuming, but which have the well known effect of raising anxiety and causing considerable stress to the average patient.

Thus, there is a widely recognized need for, and it would be highly desirable to have, a therapeutic approach downsizing of breast tumors which is less expensive to apply than conventional radiological and biological treatments, and which requires less hospital time and fewer clinic visits for the patient.

Cryogenic ablation of pathological tissues has recently come into use in a variety of contexts, for destruction of pathological tissues within body organs.

Extreme cooling of tissues disorganizes cell structures of those tissues, destroying cell functionality. Tissues thus treated by extreme cold, once they no longer retain their functional (cellular) structures, are gradually absorbed by the body in the days and weeks following a cryoablation procedure.

Simple cryoablation, however, does not comport, nor does it present a convenient opportunity for, biopsy of tissues for pathological inspection. Yet, it is a well-known need and commonly accepted medical practice to perform a biopsy of tumor tissues, to confirm a diagnosis of a tumor as benign, and/or to ascertain diagnostic information about type and characteristics of a malignant growth. Classical excision surgery, despite its various disadvantages as presented hereinabove, is advantageous in that it does present a clear and simple opportunity for taking tissue from a tumor, which samples may be used to verify a diagnosis or to provide diagnostic data. During classical excision surgery, a surgeon can easily take a tissue sample and submit it either to immediate inspection or to subsequent examination in a pathology laboratory. Existing methods for cryoablation of tumors do not, however, provide such an opportunity.

Neither would a plurality of independent biopsy and cryoablation interventions be an ideal therapy. For example, a procedural sequence in which a first intervention to perform a biopsy of tissues is followed by a second intervention to perform cryoablation of a tumor, would present two disadvantages.

A first disadvantage is that plural operations introduce ambiguity regarding the physical relationship between the biopsy site on the one hand, and the cryoablation site on the other hand. In the best of cases, the spatial relationship between the site of biopsy and the site of a subsequent independently-practiced cryoablation is known only approximately, and is subject to error and misinterpretation. If two such procedures are performed separately and independently, and particularly if they are performed on different days or by different practitioners, the resultant ambiguity may introduce a significant doubt into the relevance of a pathology report, based on a biopsy sample, to the specific tissues actually cryoablated.

A second disadvantage to plural interventions is cosmetic: two insertions rather than one may double or more than double the cosmetic damage done by the procedure, particularly in patients with a skin which tends to scar.

Thus, there is a widely recognized need for, and it would be highly desirable to have, apparatus and method for treatment of benign tumors and pre-operative treatment of malign tumors, which provides for downsizing of a tumor by cryoablation, and which also enables and facilitates extraction of tissue samples from an affected area in a form suitable for pathological examination and verification of diagnosis. It is further highly desirable that such apparatus and method require only a single incision in the breast, and that they provide accurate and unambiguous information about the relative positions of the biopsy site and the cryoablation site.

Downsizing is also practiced in various other therapeutic contexts, applied to lipomas or to normal tissue where volume reduction is desired, such as in the face, thighs, buttocks, and abdomen.

Currently mammo-reduction and lipo-reduction are performed either by conventional surgery, or by liposuction, by which excessive fat tissue is suctioned through incisions made in the skin. Open surgery, however, carries a risk of scarring. Lipo-suction also presents disadvantages, particularly inability to control sculpturing of the suctioned tissue, hazards of hematomae formation and subsequent fibrosis and deformation, and risk of fat embolism.

Thus, there is a widely felt need for, and it would be highly advantageous to have, device and method offering a minimally invasive technique for downsizing of fat tissues and other tissues, without the risks associated with lipo-suction and with open surgery.

### SUMMARY OF THE INVENTION

According to one aspect of the present invention there is provided a biopsy-enabled cryoablation device for treating a tumor of a body organ, comprising: a) an introducer having an edge shaped to enable penetration of the introducer into the organ, thereby enabling the introducer to be installed in the organ; b) a biopsy access port operable to enable passage of a biopsy tool through the introducer into tissues of the organ, thereby enabling the biopsy tool to perform biopsy sampling of the tissues when the introducer is installed in the organ; and c) a cryoprobe access port operable to enable passage of a cryoprobe through the introducer into tissues of the organ, thereby enabling the cryoprobe to cryoablate at least a portion of the tissues when the introducer is installed in the organ.

According to further features in preferred embodiments of the invention described below the introducer is shaped to enable and facilitate penetration of the introducer into a breast, and to enable and facilitate installation of the introducer in a breast.

According to still further features in the described preferred embodiments, the device further comprising a plurality of the biopsy access ports and a plurality of the cryoprobe access ports, and preferably also comprising a biopsy access channel communicating with the biopsy access port, the biopsy access channel being shaped and oriented to control direction of deployment of the biopsy tool when the biopsy tool is deployed through the biopsy access port. The device preferably also comprises a cryoprobe access channel communicating with the cryoprobe access port, the cryoprobe access channel being shaped and oriented to control direction of deployment of the cryoprobe when the cryoprobe is deployed through the cryoprobe access port.

According to still further features in the described preferred embodiments, the biopsy access port and the cryoprobe access port are a common access port, operable to enable passage of a biopsy tool through the introducer into tissues of the organ, thereby enabling the biopsy tool to perform biopsy sampling of the tissues when the introducer is installed in the organ, and further operable to enable passage of a cryoprobe through the introducer into tissues of the organ, thereby enabling the cryoprobe to cryoablate at least a portion of the tissues when the introducer is installed in the organ.

According to still further features in the described preferred embodiments, the device further comprising the cryoprobe, or a plurality of the cryoprobes, and the biopsy tool or a plurality of the biopsy tools, wherein the biopsy tool may be a biopsy needle.

According to still further features in the described preferred embodiments, the cryoprobe comprises a Joule-Thomson orifice and is operable to be cooled by Joule-Thomson cooling, and is further operable to be heated by Joule-Thomson heating, and further comprises a thermal sensor and a heat exchanging configuration for pre-cooling cooling gas prior to delivery of the cooling gas to the Joule-Thomson orifice.

According to still further features in the described preferred embodiments, the cryoprobe comprises a shape memory alloy material which displays stress induced martensite behavior when the cryoprobe is at a deployed position, and which is in a non-stress induced martensite state when the cryoprobe is positioned in the introducer prior to deployment of the cryoprobe outside the introducer. The shape memory alloy material may be an alloy of nickel titanium.

According to still further features in the described preferred embodiments, the biopsy tool comprises a shape memory alloy material, which displays stress induced martensite behavior when the biopsy tool is at a deployed position, and which is in a non-stress induced martensite state when the biopsy tool is positioned in the introducer prior to deployment of the biopsy tool outside the introducer. The shape memory alloy material may be an alloy of nickel titanium.

According to still further features in the described preferred embodiments, the introducer comprises a Joule-Thomson cooler operable to cool cooling gas prior to supply of the cooling gas to a deployed cryoprobe, a heat exchanging configuration operable to cool cooling gas prior to supply of the cooling gas to a deployed cryoprobe, and a thermal sensor.

According to another aspect of the present invention there is provided a device for treating a tumor of a body organ, comprising a) an introducer having an edge shaped to enable the introducer to penetrate into the organ; and b) an access port alternately enabling passage therethrough of: i) a biopsy tool operable to perform biopsy sampling of tissues of the organ; and ii) a cryoprobe operable to cryoablate tissues of the organ.

According to further features in preferred embodiments of the invention described below, the introducer is shaped to enable and facilitate penetration of the introducer into a breast and to enable and facilitate installation of the introducer in a breast.

According to further features in preferred embodiments of the invention described below, the device further comprising an access channel communicating with the access port, the access channel being shaped and oriented to control direction of deployment of the biopsy tool when the biopsy tool is deployed through the access port, and to control direction of deployment of the cryoprobe when the cryoprobe is deployed through the access port.

According to further features in preferred embodiments of the invention described below, the device further comprising the biopsy tool and the cryoprobe. Preferably, the biopsy tool is a biopsy needle.

According to further features in preferred embodiments of the invention described below, the cryoprobe comprises a Joule-Thomson orifice and is operable to be cooled by passage of pressurized cooling gas through the Joule-Thomson orifice.

According to still another aspect of the present invention there is provided a system for treating a tumor of an organ, comprising: a) a cryoprobe operable to cryoablate tissues of an organ; b) a biopsy tool operable to perform biopsy sampling of tissues of an organ; c) a biopsy-enabled cryoablation device which comprises: i) an introducer having an edge shaped to enable penetration of the introducer into the organ, thereby enabling the introducer to be installed in the organ; ii) a biopsy access port operable to enable passage of the biopsy tool through the introducer into tissues of the organ, thereby enabling the biopsy tool to perform biopsy sampling of the tissues when the introducer is installed in the organ; iii) a cryoprobe access port operable to enable passage of a cryoprobe through the introducer into tissues of the organ, thereby enabling the cryoprobe to cryoablate tissues of the organ when the introducer is installed in the organ; d) a gas supply module operable to supply compressed cooling gas to the cryoprobe; and c) a control module operable to control flow of gas from the gas supply module to the cryoprobe.

According to further features in preferred embodiments of the invention described below, the introducer is shaped to enable and facilitate penetration of the introducer into a breast, and to enable and facilitate installation of the introducer in a breast.

According to further features in preferred embodiments of the invention described below, the cryoprobe comprises a thermal sensor, and the control module is operable to receive data from the thermal sensor of the cryoprobe.

According to further features in preferred embodiments of the invention described below, the introducer comprises an thermal sensor, and the control module is operable to receive data from the thermal sensor of the introducer.

According to further features in preferred embodiments of the invention described below, the command module is operable to issue commands to the gas supply module based on algorithmic control functions operable to respond to user commands and to temperature data received from the thermal sensor of the cryoprobe.

According to further features in preferred embodiments of the invention described below, the command module is operable to issue commands to the gas supply module based on algorithmic control functions operable to respond to user commands and to temperature data received from the thermal sensor of the introducer.

According to further features in preferred embodiments of the invention described below, the biopsy-enabled cryoablation device comprises a plurality of the biopsy access ports.

According to further features in preferred embodiments of the invention described below, the biopsy-enabled cryoablation device comprises a plurality of the cryoprobe access ports.

According to further features in preferred embodiments of the invention described below, the biopsy-enabled cryoablation device further comprises a biopsy access channel communicating with the biopsy access port, the biopsy access channel being shaped and oriented to control direction of deployment of the biopsy tool when the biopsy tool is deployed through the biopsy access port.

According to further features in preferred embodiments of the invention described below, the biopsy-enabled cryoablation device further comprises a cryoprobe access channel communicating with the cryoprobe access port, the cryoprobe access channel being shaped and oriented to control direction of deployment of the cryoprobe when the cryoprobe is deployed through the cryoprobe access port.

According to further features in preferred embodiments of the invention described below, the biopsy access port and the cryoprobe access port are a common access port, operable to enable passage of the biopsy tool through the introducer into tissues of the organ, thereby enabling the biopsy tool to perform biopsy sampling of the tissues when the introducer is installed in the organ, and further operable to enable passage of the cryoprobe through the introducer into tissues of the organ, thereby enabling the cryoprobe to cryoablate at least a portion of the tissues when the introducer is installed in the organ.

According to further features in preferred embodiments of the invention described below, the system further comprises a plurality of the cryoprobes and a plurality of the biopsy tools. Preferably, the biopsy tool is a biopsy needle.

According to further features in preferred embodiments of the invention described below, the cryoprobe comprises a Joule-Thomson orifice and is operable to be cooled by Joule-Thomson cooling.

According to further features in preferred embodiments of the invention described below, the cryoprobe is further operable to be heated by Joule-Thomson heating.

According to further features in preferred embodiments of the invention described below, the cryoprobe further comprises a thermal sensor.

According to further features in preferred embodiments of the invention described below, the cryoprobe further comprises a heat exchanging configuration for pre-cooling cooling gas prior to delivery of the cooling gas to the Joule-Thomson orifice.

According to further features in preferred embodiments of the invention described below, the cryoprobe comprises a shape memory alloy material.

According to further features in preferred embodiments of the invention described below, the shape memory alloy material displays stress induced martensite behavior when the cryoprobe is at a deployed position.

According to further features in preferred embodiments of the invention described below, the shape memory alloy material is in a non-stress induced martensite state when the cryoprobe is positioned in the introducer prior to deployment of the cryoprobe outside the introducer.

According to further features in preferred embodiments of the invention described below, the shape memory alloy material is an alloy of nickel titanium.

According to further features in preferred embodiments of the invention described below, the biopsy tool comprises a shape memory alloy material.

According to further features in preferred embodiments of the invention described below, the shape memory alloy material displays stress induced martensite behavior when the biopsy tool is at a deployed position.

According to further features in preferred embodiments of the invention described below, the shape memory alloy material is in a non-stress induced martensite state when the biopsy tool is positioned in the introducer prior to deployment of the biopsy tool outside the introducer.

According to further features in preferred embodiments of the invention described below, the shape memory alloy material is an alloy of nickel titanium.

According to further features in preferred embodiments of the invention described below, the introducer comprises a Joule-Thomson cooler operable to cool cooling gas prior to supply of the cooling gas to a deployed cryoprobe.

According to further features in preferred embodiments of the invention described below, the introducer comprises a heat exchanging configuration operable to cool cooling gas prior to supply of the cooling gas to a deployed cryoprobe.

According to further features in preferred embodiments of the invention described below, the introducer further comprises a thermal sensor.

According to yet another aspect of the present invention there is provided a method for reducing volume of fat tissue within a selected region of a body, comprising: a) introducing into the fat tissue a cryoprobe; and b) cooling the cryoprobe to cryoablation temperatures, thereby ablating a portion of the fat tissue, thereby reducing volume of fat tissue within the selected region. The method further comprises utilizing Joule-Thomson cooling to cool the prototype to cryoablation temperatures.

According to still another aspect of the present invention there is provided a method for treating a benign tumor of an organ, comprising: a) installing in the organ, in a vicinity of the tumor, a biopsy-enabled cryoablation device, the biopsy-enabled cryoablation device comprises: i) an introducer having an edge shaped to enable penetration of the introducer into the organ, thereby enabling the introducer to be installed in the organ; ii) a biopsy access port operable to enable passage of a biopsy tool through the introducer into tissues of the organ, thereby enabling the biopsy tool to perform biopsy sampling of the tissues when the introducer is installed in the organ; and iii) a cryoprobe access port operable to enable passage of a cryoprobe through the introducer into tissues of the organ, thereby enabling the cryoprobe to cryoablate at least a portion of the tissues when the introducer is installed in the organ; b) introducing a biopsy tool through the biopsy access port into tissues of the organ in a vicinity of the tumor, and utilizing the biopsy tool to extract a tissue sample; and c) introducing a cryoprobe through the cryoprobe access port into the tissues of the organ in a vicinity of the tumor, and cooling the cryoprobe to cryoablation temperatures, thereby cryoablating tissues in a vicinity of the cryoprobe, thereby cryoablating at least a portion of the tumor.

According to still yet another aspect of the present invention there is provided a method for treating a tumor of a breast, comprising: a) installing in the breast, in a vicinity of the tumor, a biopsy-enabled cryoablation device, the biopsy-enabled cryoablation device comprises: i) an introducer having an edge shaped to enable penetration of the introducer into the breast, thereby enabling the introducer to be installed in the breast; ii) a biopsy access port operable to enable passage of a biopsy tool through the introducer into tissues of the breast, thereby enabling the biopsy tool to perform biopsy sampling of the tissues when the introducer is installed in the breast; and iii) a cryoprobe access port operable to enable passage of a cryoprobe through the introducer into tissues of the breast, thereby enabling the cryoprobe to cryoablate at least a portion of the tissues when the introducer is installed in the breast; b) introducing a biopsy tool through the biopsy access port into tissues of the breast, and utilizing the biopsy tool to extract a tissue sample; and c) introducing a cryoprobe through the cryoprobe access port into the tissues of the breast, and cooling the cryoprobe to cryoablation temperatures, thereby cryoablating tissues in a vicinity of the cryoprobe, thereby cryoablating at least a portion of the tumor.

According to further features in preferred embodiments of the invention described below, the organ is a breast and the introducer is shaped to enable and facilitate penetration of the introducer into a breast and to enable and facilitate installation of the introducer into a breast.

According to further features in preferred embodiments of the invention described below, the method further comprises conducting a pathology examination of the tissue sample.

According to still yet another aspect of the present invention there is provided a method for reducing volume of a tumor, comprising: a) introducing into an interior volume of the tumor a cryoprobe operable to cool tissues to cryoablation temperatures; and b) cooling the cryoprobe to cryoablation temperatures, thereby cryoablating tissues in a vicinity of the cryoprobe, thereby cryoablating tissues within a vicinity of the cryoprobe, thereby reducing volume of the tumor.

According to still yet another aspect of the present invention there is provided a method for reducing volume of a tumor of a breast, comprising: a) introducing into an interior volume of the tumor a cryoprobe operable to cool tissues to cryoablation temperatures; and b) cooling the cryoprobe to cryoablation temperatures, thereby cryoablating tissues in a vicinity of the cryoprobe, thereby cryoablating tissues within a vicinity of the cryoprobe, thereby reducing volume of the tumor. The method preferably further comprises utilizing Joule-Thomson cooling to cool the cryoprobe to cryoablation temperatures.

According to still yet another aspect of the present invention there is provided a method for treating a tumor of a breast, comprising: a) introducing into an interior volume of the tumor a cryoprobe operable to cool tissues to cryoablation temperatures; and b) cooling the cryoprobe to cryoablation temperatures, thereby cryoablating tissues in a vicinity of the cryoprobe, thereby destroying cellular structures of tumor tissue and leaving disorganized material remains of the tumor tissue; and c) waiting until a portion of the disorganized material remains of tumor tissue has been absorbed by the body, and volume of the tumor is thereby reduced; and d) excising remaining portions of the tumor.

The present invention successfully addresses the shortcomings of the presently known configurations by providing device and method for treatment of benign and malignant breast tumors which speeds, shortens and simplifies clinical treatment of benign tumors and of locally-confined small malignant tumors, and which speeds, shortens, and simplifies pre-operative treatment of large malignant tumors.

The present invention further successfully addresses the shortcomings of the presently known configurations by providing device and method for therapeutic treatment of benign breast tumors and of small, locally-confined malignant breast tumors, which apparatus and method are minimally invasive, less traumatic than surgical excision, which yield superior cosmetic results when compared to classical excision therapy, and which can be performed as an out-patient procedure.

The present invention further successfully addresses the shortcomings of the presently known configurations by providing device and method for pre-operative treatment of malign tumors of the breast, which treatment results in pre-operative downsizing of tumors yet does not affect lymph node status, and therefore does not present a danger of masking symptoms and thereby causing risk of misdiagnosis and inadequate treatment.

The present invention further successfully addresses the shortcomings of the presently known configurations by providing device and method for pre-operative treatment of malign tumors of the breast, which treatment results in pre-operative downsizing of tumors, yet which does not present systemic toxicity.

The present invention further successfully addresses the shortcomings of the presently known configurations by providing device and method for downsizing of breast tumors that is less expensive than conventional radiotherapy, chemotherapy, and biological therapy treatments, and requires less hospital time and fewer clinic visits than do those treatments.

The present invention further successfully addresses the shortcomings of the presently known configurations by providing device and method for ablation of tumors and/or for pre-operative downsizing of large tumors, which also enable and facilitate extraction of tissue biopsy samples from an affected area in a form suitable for pathological examination and verification of diagnosis, which require only a single incision in the breast, and which provide accurate and unambiguous information about the relative positions of the biopsy site and the cryoablation site.

The present invention further successfully addresses the shortcomings of the presently known configurations by providing device and method for downsizing of fat tissues and other tissues, without the risks associated with lipo-suction and with open surgery.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of an exemplary cryoprobe, according to an embodiment of the present invention;

FIG. 2 is a simplified schematic of a biopsy-enabled cryoablation device, according to an embodiment of the present invention;

FIG. 3 is a simplified schematic of a system comprising a pressurized gas supply module, a control module, and biopsy-enabled cryoablation device with cryoprobe and biopsy needle in deployed positions, according to an embodiment of the present invention;

FIG. 4 is a simplified flowchart presenting procedures for selecting appropriate treatment for a breast tumor, according to an embodiment of the present invention; and

FIG. 5 is a simplified flow chart of a method for treating a breast tumor, according to an embodiment of the present invention.

### DESCRIPTION OF THE PREFERRED EMBODIMENTS

The present invention is of methods for tumors of the breast, and of system and device enabling both biopsy of breast tumor tissues and cryoablation of breast tumor tissue through a common introducer requiring a single incision in a treated breast. Specifically, the present invention can be used to ablate a benign tumor or a small malignant tumor with a single treatment and without need of surgical excision. The present invention can further be used to downsize a large malignant tumor with a single treatment, thereby simplifying subsequent surgical excision and facilitating breast preservation and breast reconstruction.

The principles and operation of cryogenic treatment of breast tumors according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, xenon, and N₂O, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

The term “downsizing” with respect to breast tumors is used herein to refer to reduction in volume of a tumor as a result of a therapeutic process.

In discussion of the various figures described hereinbelow, like numbers refer to like parts.

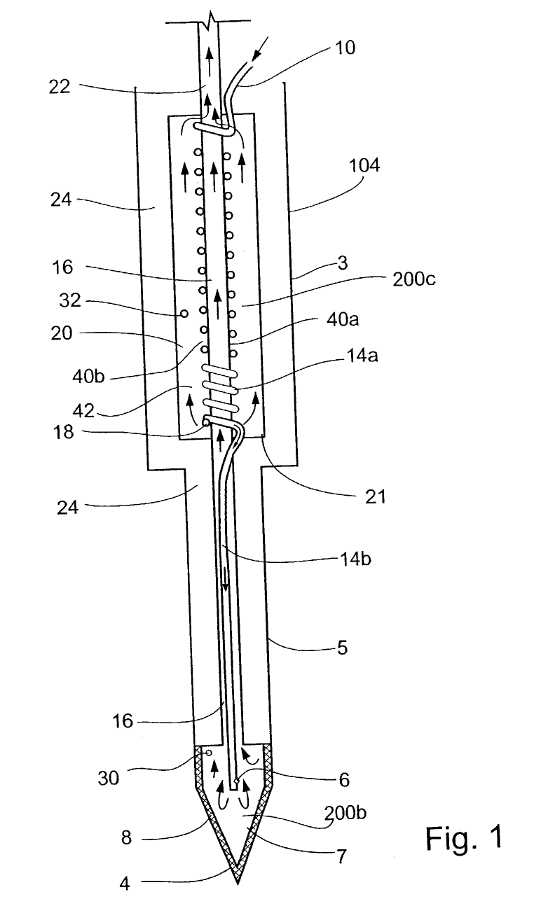

Referring now to the drawings, FIG. 1 is a simplified schematic of an exemplary cryoprobe, according to an embodiment of the present invention.

FIG. 1 illustrates an individual cryoprobe 104 according to a preferred embodiment of the present invention. Cryoprobe 104 preferably includes elongated housing 3 having a distal operating head 4 for penetrating through tissues of a patient during deployment.

Distal operating head 4 is connected to elongated housing 3 by means of an elongated member 5 substantially thin in cross section for allowing deployment into the tissues of a body. Elongated housing 3, elongated member 5, and other elements of cryoprobe 104 may include shape memory alloy, as described hereinbelow.

As shown in FIG. 1, cryoprobe 104 preferably includes a first passageway 10 extending along its length for providing gas of high-pressure to a Joule-Thomson heat exchanger 200b located at distal operating head 4, and a second passageway 16 for evacuating gas from the operating head to atmosphere. First passageway 10 is preferably in the form of a substantially thin tubular element extending along elongated housing 3, elongated member 5, and a portion of operating head 4. As shown in the figure, the portion of first passageway 10 extending along elongated housing 3 is preferably in the form of a spiral tube 14a wrapped around second passageway 16, thereby constituting a heat-exchanging configuration 40a for exchanging heat between spiral tube 14a and second passageway 16. The portion of first passageway 10 extending along elongated member 5 and portion of operating head 4 is preferably in the form of a straight tube 14b received within second passageway 16. Further as shown in the figure, tube 14b preferably penetrates into second passageway 16 substantially adjacent the connection of elongated member 5 and housing 3.

Further, elongated housing 3 preferably includes a third passageway 20 enclosing first and second passageways 10 and 16, which third passageway forms a heat-exchanging configuration 40b in the form of a heat exchanging chamber for precooling or preheating gas flowing within spiral tube 14a before it arrives to operating head 4. Third passageway 20 preferably merges with second passageway 16 at the upper end of elongated housing 3 to form a common passageway 22 for releasing gas to atmosphere.

In an alternative construction, heat exchanging configuration 40b may be formed as a porous matrix 42 filling or partially filling passageway 20, with spiral tube 14a being formed as a spiral conduit integrated into porous matrix 42 and second passageway 16 being formed as a straight conduit tunnelling through porous matrix 42.

As shown in FIG. 1, the various passageways of the device are enclosed by an insulating chamber 24 extending along housing 3 and elongated member 5.

Preferably, a device according to the present invention provides effective cooling or heating by using Joule-Thomson heat exchangers. Thus, first passageway 10 preferably includes a plurality of orifices for passage of high-pressure gas therethrough so as to cool or heat selective portions of the device, depending on the type of gas used. Gases that may be used for cooling include argon, nitrogen, air, krypton, CF₄, xenon, N₂O, or any mixture of gases, and are referred to herein as “cooling gasses”. High pressure cooling gasses are cooled by expansion when passing through a Joule-Thomson orifice, thereby providing their cooling effect. Gases that may be used for heating include helium or any mixture of gases, and are referred to herein as “heating gasses.” Heating gasses have an inversion temperature lower than temperature obtained by liquefaction of cooling gas.

According to the embodiment shown in FIG. 1, a primary Joule-Thomson heat exchanger 200b is located at distal operating head 4, which heat exchanger including: an orifice 6 located preferably at the end of straight tube 14b, and a chamber 7 defined by the inner walls of head 4. When a high-pressure cooling gas such as argon passes through orifice 6 it expands, causing it to cool and in some cases to liquify so as to form a cryogenic pool within chamber 7 of operating head 4. The cooled expanded gas, and the cryogenic pool of liquefied gas which may form, effectively cool outer sheath 8 of operating head 4. Outer sheath 8 is preferably made of a heat conducting material such as metal for effectively freezing body tissue so as to produce the desired cryoablation effect. When a high-pressure heating gas such as helium expands through orifice 6 it heats chamber 7 of operating head 4, thereby heating outer sheath 8 of the operating head. Such heating of operating head 4 may be useful to free operating head 4 from tissues to which a freezing process has caused it to adhere.

According to a preferred embodiment of the present invention cryoprobe 104 preferably includes a plurality of Joule-Thomson heat exchangers 200c for effectively precooling or preheating the gas flowing within first passageway 10. According to the embodiment shown in FIG. 1, secondary Joule-Thomson heat exchanger 200c is located within housing 3, includes a chamber 21 defined by the inner walls of passageway 20, and preferably includes an orifice 18 located preferably at the lower end of spiral tube 14a. The optional spiral construction of spiral tube 14a is designed and constructed as heat-exchanging configuration 40a, facilitating the exchange of heat between spiral tube 14a and second passageway 16, and as heat-exchanging configuration 40b facilitating the exchange of heat between spiral tube 14a and passageway 20.

When a high-pressure cooling gas such as argon passes through orifice 18 it expands and is thereby cooled. The expanded gas may liquefy so as to form a cryogenic pool within chamber 21. The cooled expanded gas, and a cryogenic pool of liquefied gas which may form, effectively cool passageway 20, thereby precooling the gas flowing within spiral tube 14a. When a high-pressure heating gas such as helium expands through orifice 18 it heats chamber 21 and passageway 20, thereby effectively preheating the gas flowing within spiral tube 14a.

Thus, gas flowing through spiral tube 14a is effectively pre-cooled or pre-heated by exchanging heat with third passageway 20. Furthermore, the gas flowing through spiral tube 14a and strait tube 14b exchanges heat with second passageway 16 which contains cooled (or heated) gas coming from operating head 4.

A cryosurgery device according to the present invention enables to effectively and quickly produce the desired freezing effect and to quickly inverse from cooling to heating so as to prevent sticking of the operating head to the tissue.

A cryosurgery device according to the present invention also enables to induce fast cyclical temperature changes in a deployed cryoprobe, such that a temperature of the probe alternates rapidly between a temperature of approximately 0° C. and a temperature below −40° C. This cryosurgical technique has been found useful in a variety of cryosurgical situations.

According to another embodiment (not shown), first passageway 10 may include a plurality of orifices located along spiral tube 14a and strait tube 14b. Further, a device according to the present invention may include a plurality of Joule-Thomson heat exchangers for cooling or heating selected portions of the device, wherein each Joule-Thomson heat exchanger includes a plurality of orifices.

The heating mechanisms heretofore described, and the cooling mechanism heretofore described, may be separate mechanisms both contained within cryoprobe 104, yet in a preferred embodiment these mechanisms are a combined heating/cooling mechanism. First passageway 10 is designed and constructed so as to be coupleable to a first gas source, supplying a high-pressure cooling gas, and also to be coupleable to a second gas source supplying high-pressure heating gas. Thus coolable cryoprobe 104 may also be heatable.

Cryoprobe 104 preferably further comprises control elements for regulating the flow of gas from the first gas source and the second gas source. In a preferred embodiment, cryoprobe 104 includes a thermal sensor 30, such as, for example, a thermocouple, for monitoring the temperature within chamber 7 of operating head 4 at the distal portion of cryoprobe 104. An additional thermal sensor 32 may also be used to monitor temperature within chamber 21, or alternatively be placed at some other convenient position within cryoprobe 104 for monitoring local temperature conditions there.

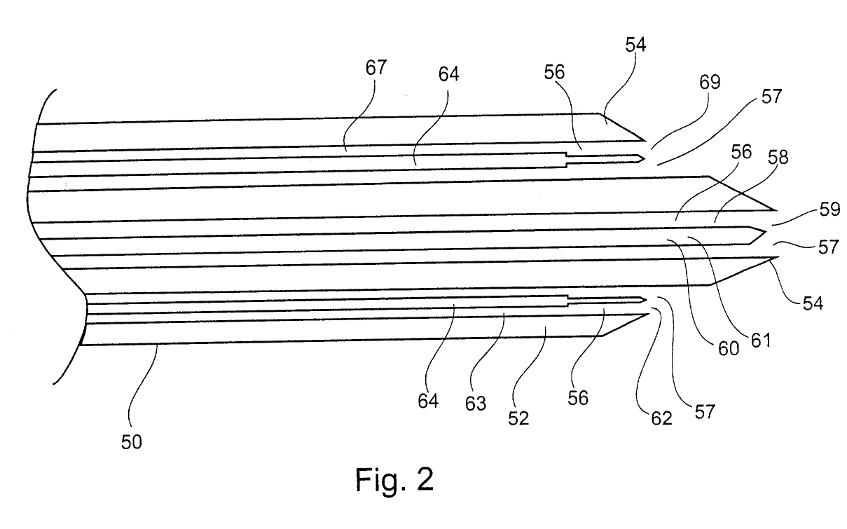

Attention is now drawn to FIG. 2, which is a simplified schematic of a biopsy-enabled cryoablation device, according to an embodiment of the present invention. FIG. 2 presents a biopsy-enabled cryoablation device 50. Cryoablation device 50 is particularly well adapted to ablating and to downsizing tumors of the breast, yet also has utility in a variety of other therapeutic applications. In particular, cryoablation device 50 is useful for cosmetic downsizing of tissues, as practiced for example in mammo-reduction and lipo-reduction.

Biopsy-enabled cryoablation device 50 comprises an introducer 52, which is a sheath having a distal edge 54 sufficiently sharp to enable penetration of introducer 52 into a body organ. In a preferred embodiment, introducer 52 is designed and constructed in a shape appropriate for enabling and facilitating partial penetration of introducer 52 into a breast, for treatment of a breast tumor. When a portion of introducer 52 is inserted into a breast or other body organ in a manner appropriate for treatment of a tumor in that organ, or for cryoablation of a selected portion of tissues of that organ, then introducer 52 is referred to in the following as having been “installed” in that organ.

Introducer 52 is shaped to enable and facilitate penetration of Introducer 52 further comprises one or more access ports 57.

In a preferred embodiment, each access port 57 communicates with an access channel 56.

In a preferred embodiment illustrated in FIG. 2, at least one access port 57 is a biopsy access port 59, designed and constructed to permit passage therethrough of a biopsy tool 60. Preferably, biopsy access port 59 communicates with a biopsy access channel 58, designed and constructed to facilitate passage of biopsy tool 60 through introducer 52 and to control direction of deployment of biopsy tool 60 through biopsy access port 59 and into body tissues surrounding introducer 52.

In an additional preferred embodiment, at least one access port 57 is a cryoprobe access port 62, designed and constructed to permit passage therethrough of a cryoprobe 64. Preferably, cryoprobe access port 62 communicates with a cryoprobe access channel 63, designed and constructed to facilitate passage of cryoprobe 64 through introducer 52, and to control direction of deployment of cryoprobe 64 through cryoprobe access port 62 and into body tissues surrounding introducer 52.

In use, introducer 52 is caused to penetrate skin and tissues of a breast (or other body organ) to a selected depth and position. Biopsy tool 60, cryoprobe 64, and optionally other surgical instruments are then enabled to pass through access channels 57 into the interior of the penetrated organ, where they are used to accomplish various therapeutic operations, at positions functionally determined by selected positioning of introducer 52.

Introducer 52 is preferably between 1 mm and 10 mm in diameter, and most preferably between 2 mm and 5 mm in diameter.

Biopsy tool 60 is preferably a biopsy needle 61. Biopsy needle 61 may be any standard biopsy needle or similar tool, such as the BARD BIOPTY Instruments and Needles, produced by R. Bard, Inc., of 730 Central Avenue, Murray Hill, N.J., 07974. Additional examples are MAGNUM Biopsy Instrument and Needles, MAX-CORE Disposable Biopsy Instrument and Needles, MONOPTY disposable Biopsy Instrument and Needles, and similar tools.

Cryoprobe 64 is preferably a cryoprobe cooled by Joule-Thomson cooling, such as cryoprobe 104 described in detail hereinabove with reference to FIG. 1. Yet, alternatively, cryoprobe 64 may be any probe operable to cool tissues to cryoablation temperatures, preferably to temperatures below −40° C., to effect cryoablation.

In a further preferred embodiment, one or more access ports 57 is a common access port 69, designed and constructed to permit passage therethrough of a cryoprobe 64 or of a biopsy tool 60. Preferably, common access port 69 communicates with a common access channel 67, designed and constructed to facilitate passage of either a cryoprobe 64 or a biopsy too 60 through introducer 52, and to control direction of deployment of cryoprobe 64 or biopsy tool 60 through common access port 69 and into body tissues surrounding introducer 52. Common access port 69 and common access channel 67 are so constructed as to allow alternating sequential passage of cryoprobe 64 and biopsy tool 60.

In a preferred use, at a first time biopsy tool 60 is passed through optional channel 67 and through port 69 into body tissues, where a biopsy is performed, after which biopsy tool 60 is withdrawn. Then, at a second time, cryoprobe 64 is passed through that same optional common channel 67 and common port 69, for performance of cryoablation. Passing both biopsy tool 60 and cryoprobe 64 through a same common port 69 and optionally through a same common channel 67 serves to ensure that both biopsy and cryoablation are performed at substantially a same position within the body tissues. In an alternate preferred construction, biopsy-enabled cryoablation device 50 is designed and constructed with a single port, a common port 69. Use of a single common port, and optionally a single common channel, enables construction of a biopsy-enabled cryoablation device of minimal diameter, particularly well suited for performing a minimally invasive biopsy and cryoablation procedure, and for producing minimal cosmetic after-effects.

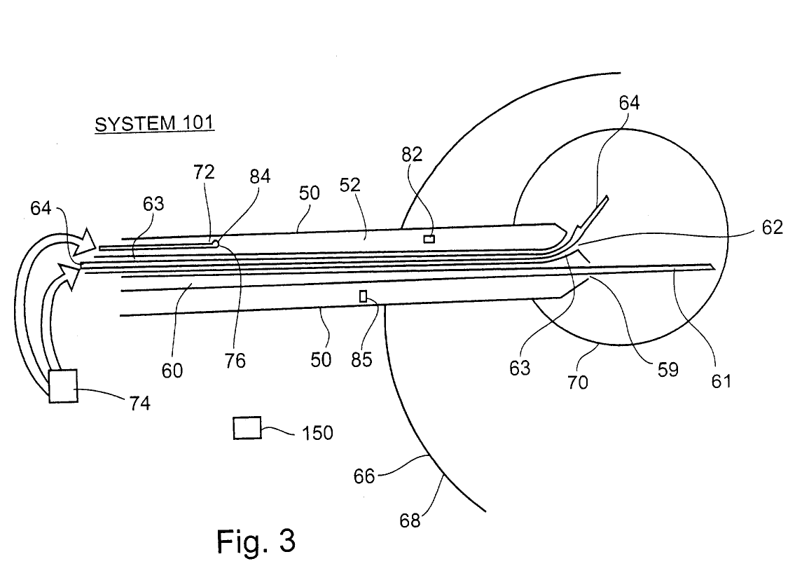

Attention is now drawn to FIG. 3, which is a simplified schematic of a system 101 comprising a biopsy-enabled cryoablation device 50, a pressurized gas supply module 74, and a control module 150. Biopsy-enabled cryoablation device 50 is shown with a cryoprobe 64 and biopsy needle 61 in deployed positions, according to an embodiment of the present invention.

In FIG. 3, biopsy-enabled cryoablation device 50 is shown having penetrated a breast 66 (or other bodily organ 68), and further having penetrated a tumor 70 within breast 66 or organ 68.

Biopsy tool 60 is shown deployed through biopsy access port 59 into tumor 70, where it may be used to perform a biopsy of tissues.

Cryoprobe 64 is shown as deployed through cryoprobe access port 62 into tumor 70, where it may be used to perform cryoablation of tissues.

In a preferred embodiment each cryoprobe 64 has a cross section of between 0.3 mm and 3 mm, and most preferably between 0.5 mm and 1.5 mm. In their undeployed, retracted, state, cryoprobes 64 will fit in the space made available for them within introducer 52, allowing introducer 52 to penetrate the body of a patient with little hindrance. Once at the site of a suspected tumor, one or more biopsy tools 60 may be deployed beyond introducer 52 to perform a biopsy, and one or more cryoprobes 64 may be deployed beyond introducer 52 to perform cryoablation.

In a preferred embodiment illustrated by FIG. 3, direction of deployment of cryoprobe 64 is controlled by cryoprobe access channel 63, which is formed in a shape and position that cause cryoprobe 64 to deploy from introducer 52 in a selected direction and orientation. As thus illustrated, access channels 57 may optionally be formed in a manner which causes cryoprobes 64 and/or biopsy tools 50 to deploy into body tissues surrounding biopsy-enabled cryoablation device 50 in selected positions and directions.

In a preferred embodiment, device 50 is thus enabled to deploy a plurality of biopsy and cryoablation tools in a selected shape and pattern. In a preferred method of utilization, various models of cryoablation device 50 may be designed and constructed, each presenting an alternate configuration of pre-selected positions and orientations for deployment of biopsy tools 60 and cryoprobes 64. A surgeon is thus enabled to select for use a biopsy-enabled cryoablation device 50 presenting a configuration best suited to a particular patient and to a particular tumor.

Thus, biopsy-enabled cryoablation device 50 may be so designed and constructed that cryoprobes 64 advance, during deployment, in a plurality of different directions. In a preferred embodiment, cryoprobes 64 expand laterally away from the introducer when deployed. As cryoprobes 64 deploy in a lateral direction away from the periphery of introducer 52, they define a three-dimensional cryoablation volume. Cooling of cryoprobes 64 so positioned results in cooling and cryoablation of a three-dimensional volume of tissue approximating, in shape and size, the tree-dimensional volume defined by deployed cryoprobes 64.

In a particularly preferred embodiment of the present invention, cryoprobes 64 are partly constructed of shape memory alloy material, such as nitinol, a nickel titanium alloy. In typical use, shape memory alloy material used in cryoprobes 64 displays stress induced martensite behavior when cryoprobe 64 is at its deployed position. Also in typical use, shape memory alloy material used in cryoprobe 64 is in a non-stress induced martensite state when cryoprobe 64 is positioned within introducer 52.

Use of shape memory material in construction of cryoprobes 64 results in each cryoprobe 64 being characterized by a particular shape, and hence a particular position with respect to introducer 52, when deployed outside of introducer 52 within the body of a patient. For example, a biopsy-enabled cryoablation device 50 of selected configuration may be introduced into breast or other organ so as to be positioned beside a tumor, and cryoprobes 64 may then be deployed substantially to one side of introducer 52, for cryoablation of a volume substantially located alongside introducer 52. Alternatively, a device 50 of different configuration may be introduced into a lesion, and cryoprobes 64 may be deployed substantially around introducer 52, for cryoablation of a volume surrounding introducer 52.

Thus, in a preferred embodiment, deployment of cryoprobes 64 creates a shaped volume of deployed cryoprobes, which may be a predefined shaped volume within the body. Deployed cryoprobes 64 are then cooled so as to perform cryoablation, resulting in a shaped volume of cryoablation.

It is a major advantage of the method of the present invention that a surgeon performing a cryoablation can cause the shape and position of the cryoablation volume substantially to conform to the shape and position of the tissues the surgeon desires to cryoablate. The method of the present invention permits cryoablation of exactly defined, preselected volumes.

Biopsy tools 60 may similarly be designed and constructed to be deployed in selected shapes and selected directions around introducer 52. It is thus a further major advantage of the method of the present invention that a surgeon performing cryoablation can extract biopsy samples of tissues both before and after cryoablation, and can further know with a high degree of exactness what spatial relationship obtained in the body, between tissues extracted as a biopsy sample, and tissue cryoablated through use of cryoprobes 64.

Biopsy-enabled cryoablation device 50 may further optionally comprise a gas pre-conditioner 72, generally used to pre-cool cooling gas destined to be utilized for cooling cryoprobes 64. Pre-conditioner 72 is preferably implemented as a Joule-Thomson heat exchanger 84 having a Joule-Thomson orifice 76 through which compressed gas, supplied through a pre-cooling gas lumen 78, is allowed to expand into introducer 52. If the pressurized gas so supplied is a cooling gas, pre-conditioner 72 pre-cools gasses transiting introducer 52 in direction of distal portions of cryoprobes 64. If the pressurized gas so supplied is a heating gas, pre-conditioner 72 pre-heats gasses transiting introducer 52 in direction of distal portions of cryoprobes 64.

Cryoablation device 50 may further optionally comprise a thermal sensor 82, operable to report temperatures within introducer 52 to a control module 150. In a preferred embodiment, control module 150 is similarly operable to receive data from sensors in other areas of BCD 50, such as from sensors 30 and 32 of a cryoprobe 64 implemented as cryoprobe 104 of FIG. 1.

Control module 150 is thus preferably operable to receive data from sensors 82, 30, and 32, and to receive commands from an operator.

In a preferred embodiment, system 101 comprises a biopsy-enabled cryoablation device 50, a gas supply module 74, and control module 150. Gas supply module 74 is operable to supply pressurized cooling gas to cryoprobes 64 and optionally to pre-cooler 72. Gas supply module 74 is preferably also operable to supply pressurized heating gas to cryoprobes 64 and optionally to gas pre-conditioner 72. Control module 150 is operable to issue commands to gas supply module 74, and gas supply module 74 is operable to respond to such commands by regulating quantities or pressures of cooling gas and/or of heating gas which are made available to cryoprobes 64 and optionally to gas pre-conditioner 72. Control module 150 preferably issues such commands to gas supply module 74 based on algorithmic control functions operable to respond to user commands and to temperature data from sensors, including temperature sensors such as sensors 30, 32, and 82, and optionally also pressure sensors 85, of device 50.

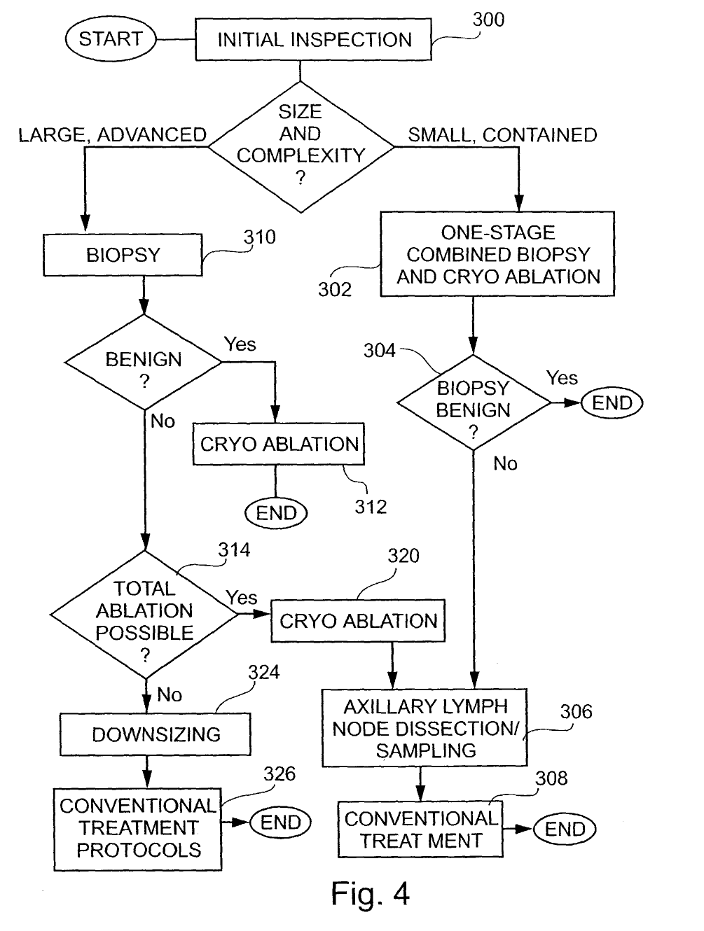

Attention is now drawn to FIG. 4, which is a simplified flowchart of procedures for selecting appropriate treatment for a breast tumor, according to an embodiment of the present invention.

Embodiments of the present invention provide various options for treating a tumor of the breast. A one-time procedure, optionally executable in an outpatient context, can provide both biopsy sampling of tumor tissue and partial or total ablation of a benign tumor. A similar one-time procedure can provide biopsy sampling of tumor tissue and total ablation of a small, well-defined and locally-contained malignant tumor. A one-time procedure can also provide significant downsizing of large or advanced stage malignant tumors, as preparatory pre-operative therapy to be followed by conventional treatment protocols. FIG. 4 presents a procedure for selection among these various forms of treatment.

At 300, a suspected tumor is inspected by palpation and/or imaging using standard imaging modalities such as mammography, ultrasound, CT, MRI, or others.

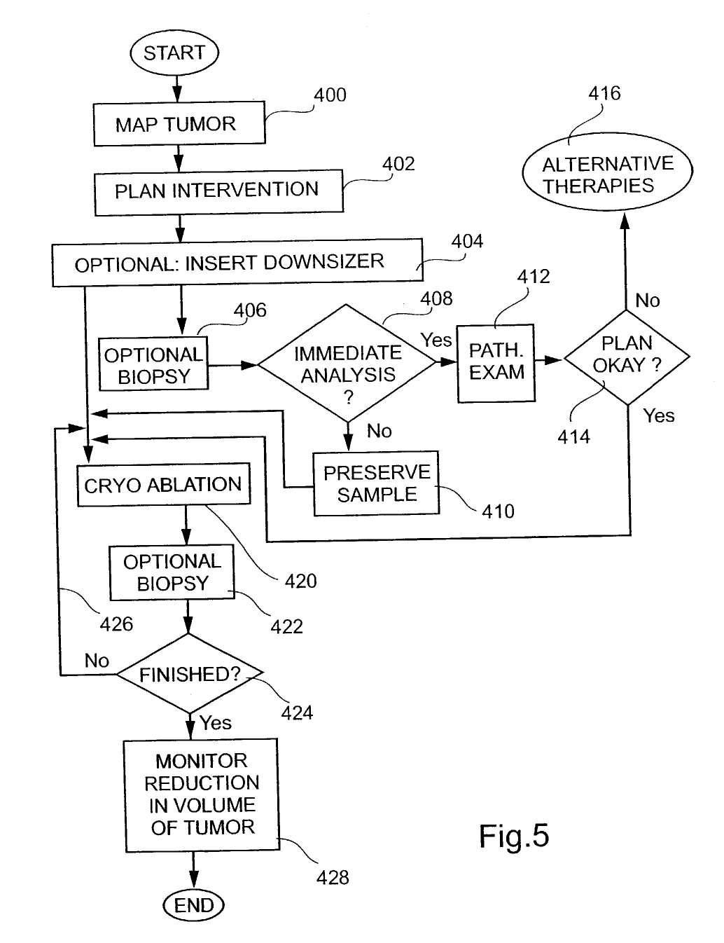

A tumor which is small, well defined, and locally confined preferably undergoes a “one-stage” combined biopsy and cryoablation procedure 302, details of which will be presented with particular reference to FIG. 5 hereinbelow. Combined procedure 302 cryoablates the tumor, while preserving a biopsy sample of tumor tissue. A tissue sample taken at 302 undergoes a pathology inspection at 304. If that tissue sample is found to be benign, the therapeutic process is complete except for normal patient follow-up. If inspection of the biopsy sample reveals that the tumor was malignant, conventional procedures for axillary lymph node dissection/sampling are undertaken at 306, and conventional treatment protocols, as suggested by results of lymph node analysis at 306, are undertaken at 308.

In a currently preferred protocol, tumors of less than 15 mm in diameter are considered sufficiently small to be recommended for combined biopsy and cryoablation procedure 302.

A tumor found at step 300 to be large, multi-focal, or to show other signs of complexity or advanced staging is inspected by biopsy at 310. A tumor found at 310 to be benign may be optionally cryoablated at 312. Cryoablation 312 may be a combined biopsy and cryoablation procedure similar to that at 302, or any other cryoablation procedure. Decision as to whether to execute optional cryoablation 312, and as to whether to execute partial or total ablation of the tumor, will depend on clinical policy, aesthetic considerations, and patient's desires.

Should the tumor be found, at step 310, to be malignant, a judgement is made at 314 as to whether total ablation of the tumor is possible, depending on such factors as tumor size, stage, position, and types of tissue involvement. If total cryoablation is deemed possible, the tumor is cryoablated at 320, followed by axillary lymph node sampling or dissection at 306, followed at 322 by such conventional treatment protocols as are suggested by the results of lymph node inspection 306.

Alternatively, if at 314 the tumor is judged not to be a candidate for total cryoablation, then cryosurgical downsizing of the tumor is practiced at 324, followed by conventional treatment protocols at 326.

Attention is now drawn to FIG. 5, which is a simplified flow chart of a method for treating a breast tumor, according to an embodiment of the present invention. The method presented by FIG. 5 is applicable to the cryoablation procedure shown in FIG. 4 as step 302, and may also be utilized for cryoablation steps 312 and 324 of FIG. 4.

At step 400, medical imaging equipment such as X-ray, fluoroscope, computerized tomography (CT), ultrasound, MRI, or other forms of imaging equipment is used to locate a suspected tumor, and to map its position, shape, and dimensions.

At step 402, an intervention is planned. Planning an intervention includes defining position, shape, and size of a volume to be cryoablated. Medical judgment must be used to choose an appropriate volume to cryoablate. In the case of some tumors, the defined volume may correspond to the shape and size of a mapped tumor. In other cases, a larger volume may be defined, to ensure total destruction of all tumor tissue. In still other cases, typically in advanced-stage malignancies, a smaller volume may be defined, so as to effect downsizing of a large tumor, the remains of which, after cryoablation and partial absorption of cryoablated tissue material, will be excised using conventional surgery.

Planning an intervention optionally includes selecting a biopsy-enabled cryoablation device configuration such that a volume defined by a set of cryoprobes 64 deployable from a selected biopsy-enabled cryoablation device corresponds to the shape and size of a defined volume desired to be cryoablated, as was discussed hereinabove with reference to FIG. 3.

Step 402 further optionally comprises selecting a biopsy-enabled cryoablation device 50 so configured that position and direction of one or more biopsy tools 60, deployed from device 50 when device 50 is inserted into a breast or other organ as planned, will extract a tissue sample from a diagnostically significant locus.

At optional step 404, a biopsy-enabled cryoablation device 50 is introduced into the body of a patient at a tumor site identified and located in step 400. In a preferred embodiment, medical imaging modalities are used guide insertion and placement of a selected device 50 into the body of a patient at a tumor site identified and located in step 400.

At optional step 406, a biopsy is performed, to extract a tissue sample for pathology analysis. Biopsy may be performed as optional step 406, or as optional step 422 (described below), or both as step 406 and as step 422.

In a preferred embodiment, biopsy 406 is performed by deploying a biopsy tool 60 through biopsy access port 59 of a device 50, as described hereinabove with particular reference to FIG. 2 and to FIG. 3.

At optional step 408, a decision is taken whether to perform immediate analysis of a biopsy sample extracted in optional step 406. If immediate analysis is not to be performed, the extracted biopsy sample is preserved for subsequent analysis in step 410. Alternatively, immediate pathological analysis is performed at step 412, and a decision taken at 414, based on results of analysis 412, as to whether the intervention planned in step 402 is considered appropriate in light of analysis results. If the planned intervention is no longer deemed appropriate, plan 402 may be revised and the procedure restarted, or alternative treatments may be undertaken, at 416.

At step 420, at least one cryoprobe is deployed into selected body tissues and cooled to cryoablation temperatures to cryoablate those selected tissues. In a preferred embodiment, at step 420 at least one cryoprobe 64 is deployed from a biopsy-enabled cryoablation device 50 already inserted in a breast or other organ at step 404, and that at least one cryoprobe 64 is cooled to cryoablation temperatures to cryoablate those selected tissues.

At optional step 422, a biopsy sample may be taken, useful to assist in evaluating results of cryoablation undertaken at step 420. This biopsy sample may be preserved for subsequent examination. Alternatively, it may be examined immediately and used to determine, at step 424, whether cryoablation process 420 is deemed to be complete. Should analysis of this biopsy sample indicate that cryoablation 420 was incomplete, cryoablation 420 may be continued or repeated, as shown by arrow 426.

If optional biopsy 422 is not taken, or if at 424 cryoablation 420 is deemed to be complete, active intervention will have terminated at this point. Monitoring the patient's status over subsequent days and weeks, at step 428, will indicate a gradual reduction in volume of the tumor, as tissue material, its internal (cellular) organization having been disrupted by the cryoablation process of step 420, is gradually absorbed into the body and carried away from the ablation site.

Thus, the procedure outlined in FIG. 5 constitutes a process whereby a single intervention, suitable to an outpatient clinic context, can provide an appropriate therapeutic response to a breast tumor.

With respect to benign tumors of the breast, the process described in FIG. 5 will in many cases suffice as a complete physical intervention and complete treatment.

With respect to well-defined and locally contained small malignant tumors, the procedure outlined in FIG. 5 may also constitute a sufficient physical intervention for treatment, possibly supplemented by additional chemotherapy and/or radiotherapy treatments, according to common practice.

With respect to large or advanced stage malignant tumors, the procedure outlined in FIG. 5 may provide significant pre-operative downsizing of a tumor, thereby facilitating a subsequent excision surgery and enhancing possibilities of breast conservation and/or breast reconstruction during excision surgery.

It is noted that methods and devices described hereinabove in the context of treatment of tumors of the breast may be useful in a variety of other therapeutic contexts. It is noted in particular that device 50, in its ability to deploy a plurality of cryoprobes in a shaped configuration, may be particularly useful for cosmetic downsizing of non-pathological tissues, such as fat tissues. Use of a cryoprobe such as cryoprobe 104 described hereinabove, or of cryoablation device 50 described hereinabove, to ablate unwanted fat tissues or other unwanted body tissues, is a recommended procedure according to a preferred embodiment of the present invention. Their use presents advantages of minimal invasiveness and little risk of scarring or of other unwanted side effects when compared to prior-art techniquest such as open surgery or lipo-suction.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A biopsy-enabled cryoablation device for treating a tumor of a body organ, comprising:
a) an introducer having an edge shaped to enable penetration of said introducer into said organ, thereby enabling said introducer to be installed in said organ;
b) a biopsy access port operable to enable passage of a biopsy tool through said introducer into tissues of said organ, thereby enabling said biopsy tool to perform biopsy sampling of said tissues when said introducer is installed in said organ; and
c) a cryoprobe access port operable to enable passage of a cryoprobe through said introducer into tissues of said organ, thereby enabling said cryoprobe to cryoablate at least a portion of said tissues when said introducer is installed in said organ.

2. The device of claim 1, wherein said introducer is shaped to enable and facilitate penetration of said introducer into a breast, and to enable and facilitate installation of said introducer in a breast.

3. The device of claim 1, further comprising a plurality of said biopsy access ports.

4. The device of claim 1, further comprising a plurality of said cryoprobe access ports.

5. The device of claim 1, further comprising a biopsy access channel communicating with said biopsy access port, said biopsy access channel being shaped and oriented to control direction of deployment of said biopsy tool when said biopsy tool is deployed through said biopsy access port.

6. The device of claim 1, further comprising a cryoprobe access channel communicating with said cryoprobe access port, said cryoprobe access channel being shaped and oriented to control direction of deployment of said cryoprobe when said cryoprobe is deployed through said cryoprobe access port.

7. The device of claim 1, wherein said biopsy access port and said cryoprobe access port are a common access port, operable to enable passage of a biopsy tool through said introducer into tissues of said organ, thereby enabling said biopsy tool to perform biopsy sampling of said tissues when said introducer is installed in said organ, and further operable to enable passage of a cryoprobe through said introducer into tissues of said organ, thereby enabling said cryoprobe to cryoablate at least a portion of said tissues when said introducer is installed in said organ.

8. The device of claim 1, further comprising said cryoprobe.

9. The device of claim 8, further comprising a plurality of said cryoprobes.

10. The device of claim 1, further comprising said biopsy tool.

11. The device of claim 10, further comprising a plurality of said biopsy tools.

12. The device of claim 10, wherein said biopsy tool is a biopsy needle.

13. The device of claim 8, wherein said cryoprobe comprises a Joule-Thomson orifice and is operable to be cooled by Joule-Thomson cooling.

14. The device of claim 13, wherein said cryoprobe is further operable to be heated by Joule-Thomson heating.

15. The device of claim 13, wherein said cryoprobe further comprises a thermal sensor.

16. The device of claim 13, wherein said cryoprobe further comprises a heat exchanging configuration for pre-cooling cooling gas prior to delivery of said cooling gas to said Joule-Thomson orifice.

17. The device of claim 8, wherein said cryoprobe comprises a shape memory alloy material.

18. The device of claim 17, wherein said shape memory alloy material displays stress induced martensite behavior when said cryoprobe is at a deployed position.

19. The device of claim 17, wherein said shape memory alloy material is in a non-stress induced martensite state when said cryoprobe is positioned in said introducer prior to deployment of said cryoprobe outside said introducer.

20. The device of claim 17, wherein said shape memory alloy material is an alloy of nickel titanium.

21. The device of claim 10, wherein said biopsy tool comprises a shape memory alloy material.

22. The device of claim 21, wherein said shape memory alloy material displays stress induced martensite behavior when said biopsy tool is at a deployed position.

23. The device of claim 21, wherein said shape memory alloy material is in a non-stress induced martensite state when said biopsy tool is positioned in said introducer prior to deployment of said biopsy tool outside said introducer.

24. The device of claim 21, wherein said shape memory alloy material is an alloy of nickel titanium.

25. The device of claim 1, wherein said introducer comprises a Joule-Thomson cooler operable to cool cooling gas prior to supply of said cooling gas to a deployed cryoprobe.

26. The device of claim 1, wherein said introducer comprises a heat exchanging configuration operable to cool cooling gas prior to supply of said cooling gas to a deployed cryoprobe.

27. The device of claim 1, wherein said introducer further comprises a thermal sensor.

28. A device for treating a tumor of a body organ, comprising
a) an introducer having an edge shaped to enable said introducer to penetrate into said organ; and
b) an access port alternately enabling passage therethrough of:
i) a biopsy tool operable to perform biopsy sampling of tissues of said organ; and
ii) a cryoprobe operable to cryoablate tissues of said organ.

29. The device of claim 28, wherein said introducer is shaped to enable and facilitate penetration of said introducer into a breast and to enable and facilitate installation of said introducer in a breast.

30. The device of claim 28, further comprising an access channel communicating with said access port, said access channel being shaped and oriented to control direction of deployment of said biopsy tool when said biopsy tool is deployed through said access port, and to control direction of deployment of said cryoprobe when said cryoprobe is deployed through said access port.

31. The device of claim 28, further comprising said biopsy tool.

32. The device of claim 28, further comprising said cryoprobe.

33. The device of claim 31, wherein said biopsy tool is a biopsy needle.

34. The device of claim 32, wherein said cryoprobe comprises a Joule-Thomson orifice and is operable to be cooled by passage of pressurized cooling gas through said Joule-Thomson orifice.

35. A system for treating a tumor of an organ, comprising:
a) a cryoprobe operable to cryoablate tissues of an organ;
b) a biopsy tool operable to perform biopsy sampling of tissues of an organ;
c) A biopsy-enabled cryoablation device which comprises:
i) an introducer having an edge shaped to enable penetration of said introducer into said organ, thereby enabling said introducer to be installed in said organ;
ii) a biopsy access port operable to enable passage of said biopsy tool through said introducer into tissues of said organ, thereby enabling said biopsy tool to perform biopsy sampling of said tissues when said introducer is installed in said organ; and
iii) a cryoprobe access port operable to enable passage of a cryoprobe through said introducer into tissues of said organ, thereby enabling said cryoprobe to cryoablate tissues of said organ when said introducer is installed in said organ;
d) a gas supply module operable to supply compressed cooling gas to said cryoprobe; and
c) a control module operable to control flow of gas from said gas supply module to said cryoprobe.

36. The system of claim 35, wherein said introducer is shaped to enable and facilitate penetration of said introducer into a breast, and to enable and facilitate installation of said introducer in a breast.

37. The system of claim 35, wherein said cryoprobe comprises a thermal sensor, and said control module is operable to receive data from said thermal sensor of said cryoprobe.

38. The system of claim 35, wherein said introducer comprises an thermal sensor, and said control module is operable to receive data from said thermal sensor of said introducer.

39. The system of claim 37, wherein said command module is operable to issue commands to said gas supply module based on algorithmic control functions operable to respond to user commands and to temperature data received from said thermal sensor of said cryoprobe.

40. The system of claim 38, wherein said command module is operable to issue commands to said gas supply module based on algorithmic control functions operable to respond to user commands and to temperature data received from said thermal sensor of said introducer.

41. The system of claim 35, wherein said biopsy-enabled cryoablation device comprises a plurality of said biopsy access ports.

42. The system of claim 35, wherein said biopsy-enabled cryoablation device comprises a plurality of said cryoprobe access ports.

43. The system of claim 35, wherein said biopsy-enabled cryoablation device further comprises a biopsy access channel communicating with said biopsy access port, said biopsy access channel being shaped and oriented to control direction of deployment of said biopsy tool when said biopsy tool is deployed through said biopsy access port.

44. The system of claim 35, wherein said biopsy-enabled cryoablation device further comprises a cryoprobe access channel communicating with said cryoprobe access port, said cryoprobe access channel being shaped and oriented to control direction of deployment of said cryoprobe when said cryoprobe is deployed through said cryoprobe access port.

45. The system of claim 35, wherein said biopsy access port and said cryoprobe access port are a common access port, operable to enable passage of said biopsy tool through said introducer into tissues of said organ, thereby enabling said biopsy tool to perform biopsy sampling of said tissues when said introducer is installed in said organ, and further operable to enable passage of said cryoprobe through said introducer into tissues of said organ, thereby enabling said cryoprobe to cryoablate at least a portion of said tissues when said introducer is installed in said organ.

46. The system of claim 35, further comprising a plurality of said cryoprobes.

47. The system of claim 35, further comprising a plurality of said biopsy tools.

48. The system of claim 35, wherein said biopsy tool is a biopsy needle.

49. The system of claim 35, wherein said cryoprobe comprises a Joule-Thomson orifice and is operable to be cooled by Joule-Thomson cooling.

50. The system of claim 49, wherein said cryoprobe is further operable to be heated by Joule-Thomson heating.

51. The system of claim 49, wherein said cryoprobe further comprises a thermal sensor.

52. The system of claim 49, wherein said cryoprobe further comprises a heat exchanging configuration for pre-cooling cooling gas prior to delivery of said cooling gas to said Joule-Thomson orifice.

53. The system claim 35, wherein said cryoprobe comprises a shape memory alloy material.

54. The system of claim 53, wherein said shape memory alloy material displays stress induced martensite behavior when said cryoprobe is at a deployed position.

55. The system claim 53, wherein said shape memory alloy material is in a non-stress induced martensite state when said cryoprobe is positioned in said introducer prior to deployment of said cryoprobe outside said introducer.

56. The system of claim 53, wherein said shape memory alloy material is an alloy of nickel titanium.

57. The system of claim 35, wherein said biopsy tool comprises a shape memory alloy material.

58. The system of claim 57, wherein said shape memory alloy material displays stress induced martensite behavior when said biopsy tool is at a deployed position.

59. The system of claim 57, wherein said shape memory alloy material is in a non-stress induced martensite state when said biopsy tool is positioned in said introducer prior to deployment of said biopsy tool outside said introducer.

60. The system of claim 57, wherein said shape memory alloy material is an alloy of nickel titanium.

61. The system of claim 35, wherein said introducer comprises a Joule-Thomson cooler operable to cool cooling gas prior to supply of said cooling gas to a deployed cryoprobe.

62. The system of claim 35, wherein said introducer comprises a heat exchanging configuration operable to cool cooling gas prior to supply of said cooling gas to a deployed cryoprobe.

63. The system of claim 35, wherein said introducer further comprises a thermal sensor.

64. A method for reducing volume of fat tissue within a selected region of a body, comprising:
a) introducing into said fat tissue a cryoprobe; and
b) cooling said cryoprobe to cryoablation temperatures, thereby ablating a portion of said fat tissue,
thereby reducing volume of fat tissue within said selected region.

65. The method of claim 64, further comprising utilizing Joule-Thomson cooling to cool said prototype to cryoablation temperatures.

66. A method for treating a benign tumor of an organ, comprising:
a) installing in said organ, in a vicinity of said tumor, a biopsy-enabled cryoablation device, said biopsy-enabled cryoablation device comprises:
i) an introducer having an edge shaped to enable penetration of said introducer into said organ, thereby enabling said introducer to be installed in said organ;
ii) a biopsy access port operable to enable passage of a biopsy tool through said introducer into tissues of said organ, thereby enabling said biopsy tool to perform biopsy sampling of said tissues when said introducer is installed in said organ; and
iii) a cryoprobe access port operable to enable passage of a cryoprobe through said introducer into tissues of said organ, thereby enabling said cryoprobe to cryoablate at least a portion of said tissues when said introducer is installed in said organ;
b) introducing a biopsy tool through said biopsy access port into tissues of said organ in a vicinity of said tumor, and utilizing said biopsy tool to extract a tissue sample; and
c) introducing a cryoprobe through said cryoprobe access port into said tissues of said organ in a vicinity of said tumor, and cooling said cryoprobe to cryoablation temperatures, thereby cryoablating tissues in a vicinity of said cryoprobe,
thereby cryoablating at least a portion of said tumor.

67. A method for treating a tumor of a breast, comprising:
a) installing in said breast, in a vicinity of said tumor, a biopsy-enabled cryoablation device, said biopsy-enabled cryoablation device comprises:
i) an introducer having an edge shaped to enable penetration of said introducer into said breast, thereby enabling said introducer to be installed in said breast;
ii) a biopsy access port operable to enable passage of a biopsy tool through said introducer into tissues of said breast, thereby enabling said biopsy tool to perform biopsy sampling of said tissues when said introducer is installed in said breast; and
iii) a cryoprobe access port operable to enable passage of a cryoprobe through said introducer into tissues of said breast, thereby enabling said cryoprobe to cryoablate at least a portion of said tissues when said introducer is installed in said breast;
b) introducing a biopsy tool through said biopsy access port into tissues of said breast, and utilizing said biopsy tool to extract a tissue sample; and
c) introducing a cryoprobe through said cryoprobe access port into said tissues of said breast, and cooling said cryoprobe to cryoablation temperatures, thereby cryoablating tissues in a vicinity of said cryoprobe,
thereby cryoablating at least a portion of said tumor.

68. The method of claim 66, where said organ is a breast and said introducer is shaped to enable and facilitate penetration of said introducer into a breast and to enable and facilitate installation of said introducer into a breast.

69. The method of claim 66, further comprising conducting a pathology examination of said tissue sample.

70. The method of claim 67, further comprising conducting a pathology examination of said tissue sample.

71. A method for reducing volume of a tumor, comprising:
a) introducing into an interior volume of said tumor a cryoprobe operable to cool tissues to cryoablation temperatures; and
b) cooling said cryoprobe to cryoablation temperatures, thereby cryoablating tissues in a vicinity of said cryoprobe,
thereby cryoablating tissues within a vicinity of said cryoprobe,
thereby reducing volume of said tumor.

72. A method for reducing volume of a tumor of a breast, comprising:
a) introducing into an interior volume of said tumor a cryoprobe operable to cool tissues to cryoablation temperatures; and
b) cooling said cryoprobe to cryoablation temperatures, thereby cryoablating tissues in a vicinity of said cryoprobe,
thereby cryoablating tissues within a vicinity of said cryoprobe,
thereby reducing volume of said tumor.

73. The method of claim 71, further comprising utilizing Joule-Thomson cooling to cool said cryoprobe to cryoablation temperatures.

74. The method of claim 72, further comprising utilizing Joule-Thomson cooling to cool said cryoprobe to cryoablation temperatures.

75. A method for treating a tumor of a breast, comprising:
a) introducing into an interior volume of said tumor a cryoprobe operable to cool tissues to cryoablation temperatures; and
b) cooling said cryoprobe to cryoablation temperatures, thereby cryoablating tissues in a vicinity of said cryoprobe, thereby destroying cellular structures of tumor tissue and leaving disorganized material remains of said tumor tissue; and
c) waiting until a portion of said disorganized material remains of tumor tissue has been absorbed by the body, and volume of said tumor is thereby reduced; and
d) excising remaining portions of said tumor.
